(19) United States
(12) Patent Application Publication
(10) Pub. No.: US 2019/0307795 A1
(43) Pub. Date: Oct. 10, 2019

(54) REGULATORY T CELLS TARGETED WITH CHIMERIC ANTIGEN RECEPTORS

(71) Applicant: The Board of Trustees of the Leland Stanford Junior University, Stanford, CA (US)

(72) Inventors: Sai-Wen Tang, San Mateo, CA (US); Panagiota Iliopoulou, San Carlos, CA (US); Magdiel Perez Cruz, San Mateo, CA (US); Everett Hurteau Meyer, Belmont, CA (US)

(21) Appl. No.: 16/256,262

(22) Filed: Jan. 24, 2019

Related U.S. Application Data
(60) Provisional application No. 62/622,304, filed on Jan. 26, 2018.

(51) Int. Cl. A61K 35/17; A61P 3/10; A61P 1/04; A61P 37/06; C07K 14/725; C07K 16/28; A61K 49/00

(52) U.S. Cl. CPC A61K 35/17; A61P 3/10; A61P 1/04; A61K 49/0043; C07K 14/7051; C07K 16/28; A61P 37/06

(57) ABSTRACT

Regulatory T cells (Treg) are engineered to express a chimeric antigen receptor (CAR), that specifically binds folate receptor beta; and are administered to an individual for treatment of inflammation at sites characterized by the presence of activated myeloid cells. Also provided are methods for utilized engineered T regulatory cells to enhance hematopoietic cell transplantation.

### CROSS REFERENCE

This application claims benefit of U.S. Provisional Patent Application No. 62/622,304, filed Jan. 26, 2018, which application is incorporated herein by reference in its entirety.

### FEDERALLY SPONSORED RESEARCH AND DEVELOPMENT

This invention was made with Government support under contract HL119590 awarded by the National Institutes of Health. The Government has certain rights in the invention.

### BACKGROUND OF THE INVENTION

The concept of introducing into a cytotoxic T-cell hybridoma the genetic material for an antibody recognizing a model antigen (a hapten, 2,4,6-trinitrophenyl) was first described in 1989. The principles have since been applied to a number of tumor antigen specificities. At its simplest embodiment, a chimeric T cell antigen receptor (CAR) is a polypeptide comprising sequences of a light and heavy chain from an antibody, linked to the signaling machinery of the T-cell receptor, typically the t chain. Modifications of this design have led to the addition of costimulatory domains that are derived from one or more of the endogenous molecules used by T cells, such as CD27, CD28, CD134, or CD137. CAR constructs typically consists of an extracellular target-binding domain, a hinge region, a trans-membrane domain that anchors the CAR to the cell membrane, and one or more intracellular signaling domains. The target-binding domain is usually derived from the light and heavy chain portions of a single chain variable fragment (scFv). Affinity and avidity are much higher for CAR binding versus binding of a T cell receptor to its cognate antigen. CARs recognize cell surface proteins, and therefore targeting is not MHC-restricted. Furthermore, unlike TCR-based recognition, CAR recognition is not dependent on processing and antigen presentation.

The incorporation of costimulatory molecules such as CD27, CD28, CD134 (OX40), CD137 (4-1BB), CD244, or ICOS into a CAR can augment the effects of t chain signaling and enhance T-cell proliferation and persistence. Incorporation of a single costimulatory molecule has been found to lead to superior persistence and other T-cell functions.

CAR constructs can be introduced into T cells using viral or non-viral techniques. Gammaretroviral or lentiviral vectors integrate into the host cell genome and have low intrinsic immunogenicity and hence lead to permanent transgene expression. Other viral vectors include adenovirus or adeno-associated virus, which provide long-term episomal transgene expression and have been shown to infect human T cells with high efficiency, but have a disadvantage of immunogenicity. Non-viral approaches include transposon/transposase systems, such as Sleeping Beauty, that can deliver a large payload with persistent high-level transgene expression. Alternatively, a DNA plasmid encoding the CAR can be transcribed in vitro, and the resulting mRNA electroporated into T cells.

T-cell trafficking is dependent upon an array of soluble factors, receptors, and adhesion molecules. Recruitment of effector T cells into the targeted microenvironment may be impeded by sub-threshold expression of homing and trafficking molecules on tumor microvessels and conversely may be enhanced by cytokine signaling. T cells can be transduced or electroporated to overexpress relevant chemokine and homing molecules to promote homing to desired tissues. See, for example Di Stasi et al. Blood 2009; 113:6392-6402; Craddock et al. J Immunother 2010, 33:780-788. Strategies to enhance T-cell persistence after transfer include exogenous cytokine administration, overexpression of pro-survival signals, or the reversal of anti-survival signals.

Further methods of treating disease with engineered T cells is of great clinical interest. The present invention addresses this issue.

### SUMMARY OF THE INVENTION

Compositions and methods are provided for treating inflammatory disease in an individual by targeting regulatory T cells to sites where activated myeloid cells, particularly macrophages, are present. Engineered regulatory T cells are provided for administration to the individual; which T cells express a chimeric antigen receptor (CAR) that binds to folate receptor beta (FRβ), thereby localizing the regulatory T cells at the sites where activated myeloid cells are present.

Engineered regulatory T cells comprising a CAR that recognizes FRβ may bind to FRβ directly or indirectly. Indirect binding systems utilize a CAR, which may be referred to as mAbCAR, that comprises as a targeting moiety an scFv that specifically binds to a non-endogenous antigenic moiety. In some embodiments, the antigenic moiety is a small molecule, e.g. a hapten, including without limitation fluorescein isothiocyanate (FITC), streptavidin, biotin, dinitrophenol, phycoerythrin (PE), and the like. Fluorescein isothiocyanate (FITC) is exemplary. An antibody conjugated to the non-endogenous antigenic moiety is administered to the individual in combination with the engineered regulatory T cells, where the mAbCAR binds to the antibodies, which are bound to FRβ expressed by activated myeloid cells. In the direct binding embodiment, the CAR comprises an scFv specific for FRβ, and thereby directly binds to the activated myeloid cells.

In some embodiments of the invention, the Treg cells are natural Treg cells. The cells may be autologous or allogeneic with respect to the recipient. In some embodiments, Treg cells are isolated from a peripheral blood sample by selection for cells that express CD4 and CD25, providing a substantially pure population of CD4⁺ CD25⁺ cells, which typically are also FoxP3⁺ cells. In some embodiments, the cells are expanded in culture following introduction of the CAR genetic construct. Alternatively, other immunoregulatory populations can be utilized, e.g. induced T regulatory cells; Lag3+, PD-1+ invariant natural killer cells; TIM-1+ regulatory B cells, and the like.

In some embodiments, the CAR construct encodes one or more costimulatory molecule(s). In some embodiments, the costimulatory molecule comprises the CD28 activating domain. In some embodiments, the CAR construct encodes or more T cell downregulatory proteins, such as an immune checkpoint protein. In some embodiments, the immune checkpoint protein is CTLA4. In some embodiments, the immune checkpoint protein is LAG3. In some embodiments both CTLA4 and LAG3 are expressed by the engineered Treg cell. In some embodiments, IL-2 pathway proteins are expressed by the engineered Treg cells.

The engineered Treg cells are administered to an individual suffering from an inflammatory condition. For the indirect binding embodiment, the engineered cells are administered in combination with administration of the folate receptor targeting antibodies. By localizing Treg cells at the site of inflammation, the effectiveness of the Treg cells is enhanced.

Highly selective targeted T cell therapies are effective non-toxic modalities for the treatment of various conditions. Inflammatory conditions in which activated monocytic cells are involved are of particular interest. For example, it has been shown that FRβ is expressed and functional in synovial macrophages in rheumatoid arthritis patients. Patients with a variety of inflammatory disorders, including rheumatoid arthritis, Crohn's disease, ischemic bowel disease, Sjogren's syndrome, localized infections, atherosclerosis, and organ transplant rejection show FRβ expression at inflammatory sites. It has been surprisingly found that engineered Treg localization can persist long after transient expression of the CAR construct. In some embodiments, expression of the mAbCAR construct by an engineered T cell is transient, e.g. detectable expression is present for less than about 4 weeks, less than about 3 weeks, less than about 2 weeks, less than about 1 week.

In some embodiments of the invention, the engineered Treg cells are administered to the individual for treatment of a diabetic condition, including insulin resistance, metabolic syndrome, obesity, Type 2 diabetes, insulin dependent diabetes mellitus (IDDM), etc. In some embodiments of the invention, the engineered Treg cells are administered to the individual for treatment of colitis, including without limitation colitis associated with inflammatory bowel disease (IBD).

In other embodiments, methods are provided for enhancing hematopoietic cell transplantation (HCT). Such transplantation may be utilized for treatment of cancer or for other conditions requiring reconstitution of the hematopoietic system. The recipient may have been treated by myeloblative conditioning prior to transplantation. A graft recipient is infused with hematopoietic cells from a donor, in combination with engineered regulatory T cells; which T cells express a chimeric antigen receptor (CAR), e.g. a mAbCAR as described above, that binds directly or indirectly to a molecule involved in hematopoietic stem cell migration or homing, which molecules include, without limitation, MHC-class I antigens, SDF-1, integrin VLA-4, VLA-5 and Rac-1. The addition of the engineered Treg cells increases chimerism of the host following engraftment.

The Treg cells may be substantially pure, where at least about 50% of the cells in the population are FOXP3⁺, at least about 75% of the cells in the population are FOXP3⁺, at least about 85% of the cells in the population are FOXP3⁺, at least about 90% of the cells in the population are FOXP3⁺, at least about 95% of the cells in the population are FOXP3⁺. The cells may be selected as described above, and modified to express the CAR construct. An effective dose of Treg may comprise up to about 10⁸ FOXP3⁺ cells/kg of recipient body weight, up to about 10⁷ FOXP3⁺ cells/kg of recipient body weight, up to about 5×10⁶ FOXP3⁺ cells/kg of recipient body weight, up to about 3×10⁶ FOXP3⁺ cells/kg of recipient body weight, up to about 10⁶ FOXP3⁺ cells/kg of recipient body weight.

In some embodiments, the hematopoietic cell sample is obtained from bone marrow, from cord blood, or by apheresis from mobilized peripheral blood. The donor may be MHC matched to the recipient. The donor may be haploidentical to the recipient. The donor may be mismatched at one or more MHC loci, e.g. mismatched at 1, 2, 3, 4, 5 or 6 of the major loci for MHC matching. The cells may be purified from the hematopoietic cell sample for expression of CD34, for example by affinity methods, including without limitation magnetic bead selection, flow cytometry, and the like from the donor hematopoietic cell sample, e.g. apheresis product. The effective dose of CD34+ cells may be from about 10⁵ to about 10⁷ CD34⁺ cells/kg of recipient body weight, and may be at least about 5×10⁵ CD34⁺ cells/kg of recipient body weight, at least about 10⁶ CD34⁺ cells/kg of recipient body weight, at least about 3×10⁶ CD34+ cells/kg of recipient body weight, at least about 5×10⁶ CD34⁺ cells/kg of recipient body weight, and may be 10⁷ CD34⁺ cells/kg of recipient body weight or more.

In some embodiments, the recipient has been treated for cancer, e.g. by treatment with a chemotherapeutic drug, radiation, etc., where the treatment may comprise myeloblative conditioning. In some embodiments the cancer is a solid tumor. In some embodiments the cancer is a leukemia or lymphoma, including without limitation, acute lymphocytic leukemia (ALL), acute myelogenous leukemia (AML), chronic myelogenous leukemia (CML), lymphomas such as Hodgkin and non-Hodgkin lymphomas, etc. In some embodiments, the tumor cells are a primary or metastatic tumor.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1C. Chimeric antigen receptor and transfection efficiency of T regulatory cells. FIG. 1A Schematic representation of CAR construct expressing the anti-FITC ScFV (1X9Q), and intramembrane CD28 and CD3 stimulatory domains. FIG. 1B Model of mAbCAR molecule expression by transfected T cells and loading with FITC-conjugated monoclonal antibody. FIG. 1C To assess the capacity of CAR Treg therapy to prevent insulin resistance, T CD4⁺CD25⁺FoxP3⁺ were transfected with CAR. 16 h after transfection efficiency were analyzed by flow cytometry. Results are expressed as representative dot plot.

FIG. 2A-2E. Treatment with CAR Treg anti-Folate receptor b antibody prevents insulin resistance and obesity in mice. FIG. 2A To assess the capacity of CAR Treg targeted with anti-folate receptor b antibody treatment to prevent insulin resistance and obesity, high or low-fat diets were provided to 4 weeks old mice to induce obesity. After 4 weeks of special diets, mice were injected with CAR Treg anti-Folate receptor b or Isotype control antibody (2×10⁶ cells/mouse; i.p.). FIG. 2B Mice body weight changes during induction of obesity. Statistic of the weight changes on week 8 was determined using the student's test. FIG. 2C Insulin resistance, FIG. 2D blood glucose level and FIG. 2E insulin production was monitored 15 days post-CAR Treg transfer. Results are expressed as mean±SEM (n≥4). *p<0.05 compared to control isotype treated-mice.

FIG. 3A-3D. Treatment with CAR Treg anti-Folate receptor b antibody prevents colitis in mice. FIG. 3A To assess the capacity of CAR Treg targeted with anti-folate receptor b antibody treatment to prevent colitis, 2.5% DSS were provided to 10 weeks old mice to induce colitis. Mice were injected with CAR Treg anti-Folate receptor b or Isotype control antibody (3×10⁶ cells/mouse; i.p.). FIG. 3B Mice body weight changes during induction of colitis. FIG. 3C Gut length, FIG. 3D Total cell count in spleen were monitored 9 days post-CAR Treg transfer. Results are expressed as mean±SEM (n≥4). *p<0.05 compared to control isotype treated-mice.

FIG. 4A-4B. FIG. 4A Effect of MHC Class I targeting on hematopoietic chimerism. FIG. 4B Effect of SDF-1 targeting on hematopoietic chimerism.

### DETAILED DESCRIPTION OF THE EMBODIMENTS

### Definitions

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The term “genetic modification” means any process that adds, deletes, alters, or disrupts an endogenous nucleotide sequence and includes, but is not limited to viral mediated gene transfer, liposome mediated transfer, transformation, transfection and transduction, e.g., viral mediated gene transfer such as the use of vectors based on DNA viruses such as lentivirus, adenovirus, retroviruses, adeno-associated virus and herpes virus.

“Variant” refers to polypeptides having amino acid sequences that differ to some extent from a native sequence polypeptide. Ordinarily, amino acid sequence variants will possess at least about 80% sequence identity, more preferably, at least about 90% homologous by sequence. The amino acid sequence variants may possess substitutions, deletions, and/or insertions at certain positions within the reference amino acid sequence.

“Antibody-dependent cell-mediated cytotoxicity” and “ADCC” refer to a cell-mediated reaction in which nonspecific cytotoxic cells that express Fc receptors, such as natural killer cells, neutrophils, and macrophages, recognize bound antibody on a target cell and cause lysis of the target cell. ADCC activity may be assessed using methods, such as those described in U.S. Pat. No. 5,821,337.

As used herein, the term “subject” denotes a mammal, such as canines; felines; equines; bovines; ovines; etc. and primates, particularly humans. Animal models, particularly small mammals, e.g. murine, lagomorpha, etc. can be used for experimental investigations. Preferably a subject according to the invention is a human.

A “cytokine” is a protein released by one cell to act on another cell as an intercellular mediator.

“Non-immunogenic” refers to a material that does not initiate, provoke or enhance an immune response where the immune response includes the adaptive and/or innate immune responses.

The term “gene” means the segment of DNA involved in producing a polypeptide chain; it includes regions preceding and following the coding region “leader and trailer” as well as intervening sequences (introns) between individual coding segments (exons). Some genes may be developed which lack, in whole or in part, introns. Some leader sequences may enhance translation of the nucleic acid into polypeptides.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

Regulatory T Cells.

Regulatory T cells (“Treg”) are a specialized subpopulation of T cells that suppress activation of the immune system and thereby maintain tolerance to self-antigens. There are various types of regulatory T cells. The majority of recent research has focused on TCRαβ+CD4+ regulatory T cells. These include natural regulatory T cells (nTreg), which are T cells produced in the thymus and delivered to the periphery as a long-lived lineage of self-antigen-specific lymphocytes; and induced regulatory T cells (iTreg), which are recruited from circulating lymphocytes and acquire regulatory properties under particular conditions of stimulation in the periphery. Both cell types are CD4+CD25+, both can inhibit proliferation of CD4+CD25− T cells in a dose dependent manner, and both are anergic and do not proliferate upon TCR stimulation. In addition to being positive for CD4 and CD25, regulatory T cells are positive for the transcription factor Foxp3, an intracellular marker.

In the methods of the invention, Treg cells can be isolated from a patient sample by selection for the phenotype of interest, e.g. for CD4+CD25+ cells. It will be understood by those of skill in the art that the stated expression levels reflect detectable amounts of the marker protein on the cell surface. A cell that is negative for staining (the level of binding of a marker specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive”. Any suitable method can be used, e.g. flow cytometry, panning, magnetic bead selection, and the like as known in the art. The cells can be expanded in culture before or after introduction of the CAR construct, e.g. by culture with anti-CD3 antibodies (for TCR stimulation) and excess exogenous IL-2 (a T cell growth factor). The anergic state of regulatory T cells can be overcome by anti-CD28 costimulation or interaction with mature dendritic cells.

In some embodiments, methods of inducing the proliferation of regulatory T cells are employed to produce an enriched population of engineered Treg cells. By an “enriched population of regulatory T cells”, it is meant that the representation of regulatory T cells in the cell population is greater than would otherwise be, e.g., in the absence of the methods provided. In other words, methods of the invention increase the percentage of regulatory T cells in the population by at least 1.5-fold or more, e.g. 2-fold or more, in some instances 3-fold or more, relative to the number of regulatory T cells that would exist in the cell population in the absence of enrichment.

Folate receptor beta (FOLR2, FRβ) is a member of the folate receptor family. Members of this gene family have a high affinity for folic acid and for several reduced folic acid derivatives, and they mediate delivery of 5-methyltetrahydrofolate to the interior of cells. This protein has a 68% and 79% sequence homology with the FOLR1 and FOLR3 proteins, respectively.

In monocytes and macrophage-lineage cells, also referred to herein as myeloid cells, FRβ expression is increased upon activation, i.e. in activated myeloid cells. The β isoform of the FR is primarily, if not exclusively, expressed on the monocyte subpopulation of human peripheral blood cells. FR-β⁺ peripheral blood cells belong almost exclusively to a proinflammatory subpopulation of monocytes, although not all proinflammatory monocytes express FR-β. For example, FRβ is expressed and functional in synovial macrophages in rheumatoid arthritis patients. Patients with a variety of inflammatory disorders, including rheumatoid arthritis, Crohn's disease, ischemic bowel disease, Sjogren's syndrome, localized infections, atherosclerosis, and organ transplant rejection show uptake of a folate at sites of inflammation (see Low et al., Acc Chem Res. 41(1):120-9 (2008); Matteson et al., Clin Exp Rheumatol 27:253-259 (2009); and Ayala-Lopez et al., J Nuc Med, 51:768-774 (2010)). These findings indicate expression of FRβ in activated macrophages and monocytes.

Macrophages are highly adaptive myeloid cells that respond to environmental stimuli and differentiate into a diversity of subsets with both pro- and anti-inflammatory properties. Although macrophages display a true continuum of phenotypes in vivo, for simplicity, these phenotypes have often been categorized into classically activated macrophages (CAMs or M1) and alternatively activated macrophages (AAMs or M2). CAMs have been linked to the development of autoimmune diseases, such as RA, diabetes, psoriasis, and Crohn's disease, AAMs have been more closely tied to tumor progression, certain allergies, and various forms of fibrosis. Based on their prominent contributions to these pathologies, activated macrophages have become a prime target for treatment of many autoimmune and inflammatory diseases.

Antibodies that specifically bind to FRβ are known in the art, or can be generated using art-recognized methods. For example, functional recombinant human FRβ protein was produced in insect cells and used as antigen to isolate the antibody m909, from a human naïve Fab phage display library. See, for example, Feng et al. Arthritis Res Ther. 2011; 13(2):R59. The anti-human FRβ antibody 94b/FOLR2 is described by Nagayoshi et al. 2005. Arthritis Rheum. 9:2666. Additional anti-human folate receptor beta antibodies and methods of use are described in U.S. Pat. No. 8,871,206; each of which are herein specifically incorporated by reference. For analysis in an animal model, for example a mouse model, antibodies specific for the mouse FRβ may find use, e.g. the CL10 antibody.

Targeting Antibody.

A targeting antibody specifically binds to the targeting antigen found in the region (or lesion) of the undesirable inflammation, i.e. folate receptor β. A targeting antibody is typically modified by conjugation to a non-endogenous antigenic moiety. For engraftment of hematopoietic cells, a targeting antibody may bind to SDF-1, MHC Class I antigens, etc.

In some embodiments the targeting antigen is an adhesion molecule involved in leukocyte trafficking, e.g. an integrin, a selectin, etc. Of interest are adhesion molecules expressed on endothelial cells, e.g. MADCAM-1, ICAM-1, VCAM-1, vascular adhesion protein 1 (VAP-1), fibronectin, paxillin, etc. Also included are monoclonal antibodies having specificity for an integrin, e.g. a β2 integrin, integrin, a β7 integrin, an αI, αM, αX, αD, α4, α9, αv, etc.

Non-Endogenous Antigenic Moiety.

A non-endogenous antigenic moiety is an antigen not normally present in the body. Typically, the moiety is of a sufficiently small size that it can be used as a label, or tag, on a monoclonal antibody, but is of a size sufficient to bind to the mabCAR protein.

In some embodiments, the antigenic moiety is a small molecule, e.g. a hapten, including without limitation fluorescein isothiocyanate (FITC), streptavidin, biotin, dinitrophenol, phycoerythrin (PE), green fluorescent protein, horseradish peroxidase, histidine, streptavidin, fluorescent tags, and the like.

The antigenic moiety may be conjugated to the targeting antibodies using techniques such as chemical coupling and chemical cross-linkers. Alternatively, polynucleotide vectors can be prepared that encode the targeting antibodies as fusion proteins.

Antibody:

As used herein, the term “antibody” refers to a polypeptide that includes canonical immunoglobulin sequence elements sufficient to confer specific binding to a particular target antigen. As is known in the art, intact antibodies produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure.

The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)2, Fab, Fv, rIgG, and scFv fragments. The term scFv refers to a single chain Fv antibody in which the variable domains of the heavy chain and of the light chain of a traditional two chain antibody have been joined to form one chain.

The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

Any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, antibody sequence elements are humanized, primatized, chimeric, etc, as is known in the art.

The use of a single chain variable fragment (scFv) is of particular interest for developing a CAR construct. scFvs are recombinant molecules in which the variable regions of light and heavy immunoglobulin chains encoding antigen-binding domains are engineered into a single polypeptide. Generally, the V_{H} and V_{L} sequences are joined by a linker sequence. See, for example, Ahmad (2012) Clinical and Developmental Immunology Article ID 980250, herein specifically incorporated by reference.

As used herein, a “vector” is any agent capable of delivering or maintaining nucleic acids to a host cell, and includes viral vectors (e.g. retroviral vectors, lentiviral vectors, adenoviral vectors, or adeno-associated viral vectors), plasmids, naked nucleic acids, nucleic acids complexed with polypeptide or other molecules and nucleic acids immobilized onto solid phase particles. The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art. Transcription of the DNA encoding the polypeptides of the present invention by higher eukaryotes is increased by inserting an enhancer sequence into the vector. Enhancers are cis-acting elements of DNA, usually about from 10 to 300 by that act on a promoter to increase its transcription. Examples including the SV40 enhancer on the late side of the replication origin by 100 to 270, a cytomegalovirus early promoter enhancer, the polyoma enhancer on the late side of the replication origin, and adenovirus enhancers. Non-integrating lentiviral vectors (NILVs) have been developed to reduce insertional mutagenesis-induced genotoxicity by eliminating the integrase function of LV vectors. Cells transduced with a NILV have double-stranded DNA circles accumulated inside the nucleus facilitating transgene expression. For transient expression, cells may be micro-injected with mRNA encoding the CAR construct.

Chimeric Antigen Receptor (CAR).

The CAR architecture may be any suitable architecture, as known in the art. The antigen recognition domain is typically derived from an scFv, which as described above may selectively bind to the non-endogenous antigenic moiety; or may selectively bind to FRβ. Exemplary anti-FRβ antibodies are described above, including for example anti-FITC scFv 1x9Q.

In certain embodiments, a cytoplasmic signaling domain, such as those derived from the T cell receptor ζ-chain, is employed as at least part of the chimeric receptor in order to produce stimulatory signals for T lymphocyte proliferation and effector function following engagement of the chimeric receptor with the target antigen. Examples include, but are not limited to, endodomains from co-stimulatory molecules such as CD28, 4-1BB, and OX40 or the signaling components of cytokine receptors such as IL7 and IL15. In particular embodiments, co-stimulatory molecules are employed to enhance the activation, proliferation, and activity of Treg cells produced by the CAR after antigen engagement. In specific embodiments, the co-stimulatory molecules are CD28, OX40, and 4-1BB and cytokine and the cytokine receptors are IL7 and IL15. The CAR may be first generation, second generation, or third generation CAR, in which signaling is provided by CD3ζ together with co-stimulation provided by CD28 and a tumor necrosis factor receptor (TNFr), such as 4-1BB or OX40), for example.

Spacer.

A spacer region links the antigen binding domain to the transmembrane domain. It should be flexible enough to allow the antigen binding domain to orient in different directions to facilitate antigen recognition. The simplest form is the hinge region from an immunoglobulin, e.g. the hinge from any one of IgG1, IgG2a, IgG2b, IgG3, IgG4, particularly the human protein sequences. Alternatives include the CH₂CH₃ region of immunoglobulin and portions of CD3. For many scFv based constructs, an IgG hinge is effective.

The length of the DNA linker used to link the scFv and zeta chain is important for proper folding. It has been estimated that the peptide linker must span 3.5 nm (35 Å) between the carboxy terminus of the variable domain and the amino terminus of the other domain without affecting the ability of the domains to fold and form an intact antigen-binding site. Many such linkers are known in the art, for example flexible linkers comprising stretches of Gly and Ser residues. The linkers used in the present invention include, without limitation, a rigid linker. In some specific embodiments of the invention, a rigid linker has the sequence SEQ ID NO:1 (EAAAK)n, where n is 1, 2, 3, 4, 5, 6, etc. In some specific embodiments, n is 3.

T2A Peptide.

T2A peptide can be used to link the CAR of the invention to an epitope tag or other protein or peptide, including without limitation a sortable tag. T2A-linked multicistronic vectors can be used to express multiple proteins from a single open reading frame. The small T2A peptide sequences, when cloned between genes, allow for efficient, stoichiometric production of discrete protein products within a single vector through a novel “cleavage” event within the T2A peptide sequence. Various 2A peptide sequences are known and used in the art, for example see Szymczak-Workman et al. (2012) Cold Spring Harb Protoc. 2012(2):199-204, herein specifically incorporated by reference. They are small (18-22 amino acids) and have divergent amino-terminal sequences, which minimizes the chance for homologous recombination and allows for multiple, different 2A peptide sequences to be used within a single vector.

Immune Responsiveness Modulators.

Immune checkpoint proteins are immune inhibitory molecules that act to decrease immune responsiveness toward a target cell. Expression of certain checkpoint proteins is frequently associated with Treg cells, e.g. CTLA4, GITR, LAGS, etc. The expression of these and other immune suppressive proteins may be upregulated in the engineered Treg cells of the invention, including upregulation by introduction of coding sequences for the proteins.

Cytotoxic T-lymphocyte-associated antigen 4 (CTLA4; also known as CD152) and programmed cell death protein 1 (PD1; also known as CD279)—are both inhibitory receptors. CTLA4 is expressed exclusively on T cells where it primarily regulates the amplitude of the early stages of T cell activation. CTLA4 counteracts the activity of the T cell co-stimulatory receptor, CD28. CD28 and CTLA4 share identical ligands: CD80 (also known as B7.1) and CD86 (also known as B7.2). The major physiological roles of CTLA4 are downmodulation of helper T cell activity and enhancement of regulatory T (TReg) cell immunosuppressive activity.

Other immune-checkpoint proteins are PD1 and PDL1. The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. PD1 is highly expressed on T_{Reg} cells, where it may enhance their proliferation in the presence of ligand.

Lymphocyte activation gene 3 (LAG3; also known as CD223), 2B4 (also known as CD244), B and T lymphocyte attenuator (BTLA; also known as CD272), T cell membrane protein 3 (TIM3; also known as HAVcr2), adenosine A2a receptor (A2aR) and the family of killer inhibitory receptors have each been associated with the inhibition of lymphocyte activity and in some cases the induction of lymphocyte anergy.

LAG3 is a CD4 homolog that enhances the function of T_{Reg} cells. LAG3 also inhibits CD8⁺ effector T cell functions independently of its role on T_{Reg} cells. The only known ligand for LAG3 is MHC class II molecules, which are expressed on tumor-infiltrating macrophages and dendritic cells. LAG3 is one of various immune-checkpoint receptors that are coordinately upregulated on both T_{Reg} cells and anergic T cells.

BTLA is an inhibitory receptor on T cells that interacts with TNFRSF14. The system of interacting molecules is complex: CD160 (an immunoglobulin superfamily member) and LIGHT (also known as TNFSF14), mediate inhibitory and co-stimulatory activity, respectively. Signaling can be bidirectional, depending on the specific combination of interactions.

A2aR, the ligand of which is adenosine, inhibits T cell responses, in part by driving CD4⁺ T cells to express FOXP3 and hence to develop into T_{Reg} cells.

Inflammatory Disease.

Inflammation is a process whereby the immune system responds to infection or tissue damage. Inflammatory disease results from an activation of the immune system that causes illness, in the absence of infection or tissue damage, or at a response level that causes illness. Inflammatory disease includes autoimmune disease, which are any disease caused by immunity that becomes misdirected at healthy cells and/or tissues of the body. For the purposes of the present invention, inflammatory diseases of interest have a detectable increase of activated myeloid cells at the sites of lesions, e.g. an increase of at least about 5%, at least about 10%, at least about 20%, at least about 50%, or more, relative to the level of activated myeloid cells in the absence of disease. The increase in activated myeloid cells can be determined by measuring, for example, uptake and visualization of labeled folate, expression of FRβ, and the like.

The immune system employs a highly complex mechanism designed to generate responses to protect mammals against a variety of foreign pathogens while at the same time preventing responses against self-antigens. In addition to deciding whether to respond (antigen specificity), the immune system must also choose appropriate effector functions to deal with each pathogen (effector specificity). A cell critical in mediating and regulating these effector functions are CD4⁺ CD25⁺ T regulatory cells. The methods provided herein allow specific targeting to Treg cells to sites of inflammation.

Included in inflammatory disease is diabetes, which is a metabolic disease that occurs when the pancreas does not produce enough of the hormone insulin to regulate blood sugar (“type 1 diabetes mellitus”) or, alternatively, when the body cannot effectively use the insulin it produces (“type 2 diabetes mellitus”).

According to recent estimates by the World Health Organization, more than 200 million people worldwide have diabetes, of which 90% suffer from type 2 diabetes mellitus. Typical long-term complications include development of neuropathy, retinopathy, nephropathy, generalized degenerative changes in large and small blood vessels and increased susceptibility to infection. Since individuals with type 2 diabetes still have a residual amount of insulin available in contrast to type 1 diabetic individuals, who completely lack the production of insulin, type 2 diabetes only surfaces gradually and is often diagnosed several years after onset, once complications have already arisen.

Insulin resistance occurs in 25% of non-diabetic, non-obese, apparently healthy individuals, and predisposes them to both diabetes and coronary artery disease. Hyperglycemia in type II diabetes is the result of both resistance to insulin in muscle and other key insulin target tissues, and decreased beta cell insulin secretion. Longitudinal studies of individuals with a strong family history of diabetes indicate that the insulin resistance precedes the secretory abnormalities. Prior to developing diabetes these individuals compensate for their insulin resistance by secreting extra insulin. Diabetes results when the compensatory hyperinsulinemia fails. The secretory deficiency of pancreatic beta cells then plays a major role in the severity of the diabetes.

Insulin resistance, typically defined as decreased sensitivity or responsiveness to the metabolic actions of insulin, is a precursor of the metabolic syndrome and type 2 diabetes. The gold standard for assessing insulin resistance in humans is the hyperinsulinemic-euglycemic clamp. This procedure assumes that at high doses of insulin infusion (>80 mU/m²·min), the hyperinsulinemic state is sufficient to completely suppress hepatic glucose production and that there is no net change in blood glucose concentrations under steady-state conditions. Under such conditions, the rate of glucose infused is equal to the rate of whole-body glucose disposal (GDR) or metabolizable glucose (M) and reflects the amount of exogenous glucose necessary to fully compensate for the hyperinsulinemia. GDR is expressed as a function of metabolic body size, such as body weight (kg), body surface area (m²; BSA), fat-free mass (kg; FFM), or metabolic size (kgFFM+17.7). Individual insulin resistance may estimated with good sensitivity (89%) and specificity (67%) from the homeostasis model assessment of insulin resistance (HOMA-IR) >5.9 or 2.8<HOMA-IR<5.9 with HDL<51 mg/dL. A conservative definition for insulin resistance may use an M value<4.7 mg/kg·min.

Insulin resistant, non-diabetic individuals have a much higher risk for developing type II diabetes than insulin sensitive subjects. However, even without developing hyperglycemia and diabetes, these insulin resistant individuals pay a significant price in terms of general health. Insulin resistance results in an increased risk for having elevated plasma triglycerides (TG), lower high-density lipoproteins (HDL), and high blood pressure, a cluster of abnormalities that have been termed by different investigators as either Syndrome X, the insulin resistance syndrome, or the metabolic syndrome. It is believed that either the hyperinsulinemia, insulin resistance, or both play a direct role in causing these abnormalities. Data from ethnic, family, and longitudinal studies suggest that a major component of resistance is inherited.

Type 2 diabetes mellitus is a multigenic disease; its already high prevalence in adults worldwide and its increasing prevalence in both adults and children indicate an urgent need to assess the genetic susceptibility to this disease as early in life as possible and to identify therapeutic interventions to prevent the onset of type 2 diabetes mellitus and to treat the disease.

Human insulin-dependent diabetes mellitus (IDDM) is a disease characterized by autoimmune destruction of the β cells in the pancreatic islets of Langerhans. An animal model for the disease is the non-obese diabetic (NOD) mouse, which develops autoimmunity. NOD mice spontaneously develop inflammation of the islets and destruction of the β cells, which leads to hyperglycemia and overt diabetes. Both CD4+ and CD8+ T cells are believed to be required for diabetes to develop: CD4+ T cells appear to be required for initiation of insulitis, cytokine-mediated destruction of β cells, and probably for activation of CD8+ T cells. The CD8+ T cells in turn mediate β cell destruction by cytotoxic effects such as release of granzymes, perforin, TNF α and IFN γ.

The depletion of β cells results in an inability to regulate levels of glucose in the blood. Overt diabetes occurs when the level of glucose in the blood rises above a specific level, usually about 250 mg/dl. In humans, a long presymptomatic period precedes the onset of diabetes. During this period, there is a gradual loss of pancreatic β cell function. The disease progression may be monitored in individuals diagnosed by family history and genetic analysis as being susceptible. The most important genetic effect is seen with genes of the major histocompatibility locus (IDDM1), although other loci, including the insulin gene region (IDDM2) also show linkage to the disease (see Davies et al, supra and Kennedy et al. (1995) Nature Genetics 9:293-298).

Markers that may be evaluated during the presymptomatic stage are the presence of insulitis in the pancreas, the level and frequency of islet cell antibodies, islet cell surface antibodies, aberrant expression of Class II MHC molecules on pancreatic β cells, glucose concentration in the blood, and the plasma concentration of insulin. An increase in the number of T lymphocytes in the pancreas, islet cell antibodies and blood glucose is indicative of the disease, as is a decrease in insulin concentration. After the onset of overt diabetes, patients with residual β cell function, evidenced by the plasma persistence of insulin C-peptide, may also benefit from the subject treatment, to prevent further loss of function.

The subject therapy will desirably be administered during the presymptomatic or preclinical stage of the disease, and in some cases during the symptomatic stage of the disease. Early treatment is preferable, in order to prevent the loss of function associated with inflammatory tissue damage. The presymptomatic, or preclinical stage will be defined as that period not later than when there is T cell involvement at the site of disease, e.g. islets of Langerhans, synovial tissue, thyroid gland, etc., but the loss of function is not yet severe enough to produce the clinical symptoms indicative of overt disease. T cell involvement may be evidenced by the presence of elevated numbers of T cells at the site of disease, the presence of T cells specific for autoantigens, the release of performs and granzymes at the site of disease, response to immunosuppressive therapy, etc.

Inflammatory Bowel Disease.

As used herein, the term “inflammatory bowel disease” or “IBD” refers to any of a variety of diseases characterized by inflammation of all or part of the intestines (e.g., colon and/or small intestine). Non-limiting examples of “IBD” include: Crohn's Disease, Ulcerative Colitis, radiation colitis, Collagenous colitis, Lymphocytic colitis, Ischaemic colitis, Diversion colitis, Behçet's disease, and Indeterminate colitis. As will be understood by one of ordinary skill in the art, the two IBD types that account for the majority of IBD clinical cases are Crohn's Disease and Ulcerative Colitis. While IBD symptoms vary from patient to patient and some may be more common than others, the symptoms can include weight loss, colon thickening, soft/loose stool (e.g., diarrhea, watery diarrhea, etc.), rectal bleeding (e.g., bloody stool), abdominal cramps, abdominal pain, vomiting, acute right lower quadrant pain, malaise, fatigue, fever, and/or anemia.

Different forms of IBD differ in the location and nature of the inflammatory changes. For example, Crohn's disease can affect any part of the gastrointestinal tract, from mouth to anus, although a majority of the cases start in the terminal ileum. Crohn's disease can also affect the entire thickness of the bowel wall. In addition, in Crohn's disease, the inflammation of the intestine can “skip” leaving normal areas in between patches of diseased intestine (sometimes referred to as skip lesions). In more severe cases, Crohn's can lead to tears (fissures) in the lining of the anus, which may cause pain and bleeding, especially during bowel movements. Inflammation may also cause a fistula to develop. In contrast, ulcerative colitis is restricted to the colon and the rectum. Microscopically, ulcerative colitis is restricted to the mucosa (epithelial lining of the gut), while Crohn's disease can affect the whole bowel wall (“transmural lesions”). In patients with ulcerative colitis, the lining of the colon can become inflamed and develop tiny open sores, or ulcers, that produce pus and mucous. The combination of inflammation and ulceration can cause abdominal discomfort and frequent emptying of the colon. Crohn's disease and ulcerative colitis can present with extra-intestinal manifestations (e.g., liver problems, arthritis, skin manifestations, eye problems, etc.). Rarely, a definitive diagnosis of neither Crohn's disease nor ulcerative colitis can be made because of idiosyncrasies in the presentation. In this case, a diagnosis of indeterminate colitis may be made.

IBD is associated with inflammation of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Chronic inflammation can also typically characterized by periods of spontaneous remission and spontaneous occurrence. “Mucosal layer of the gastrointestinal tract” is meant to include mucosa of the bowel (including the small intestine and large intestine), rectum, stomach (gastric) lining, oral cavity, and the like.

“Chronic IBD” refers to IBD that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic IBD may be expected to require a long period of supervision, observation, or care.

In some embodiments of the invention, an individual is diagnosed with a chronic inflammatory disease of the bowels prior to administration of FZHY. In some embodiments, a patient is diagnosed with ulcerative colitis. In other embodiments, a patient is diagnosed with Crohn's disease.

Diagnosis is suggested by typical symptoms and signs, particularly when accompanied by extraintestinal manifestations or a history of previous similar attacks. UC should be distinguished from Crohn disease but more importantly from other causes of acute colitis (eg, infection; in elderly patients, ischemia). In all patients, stool cultures for enteric pathogens should be done, and Entamoeba histolytica should be excluded by examination of fresh stool specimens. Sigmoidoscopy allows visual confirmation of colitis and permits direct sampling of stool or mucus for culture and microscopic evaluation, as well as biopsy of affected areas. Although visual inspection and biopsies may be nondiagnostic, because there is much overlap in appearance among different types of colitis, acute, self-limited, infectious colitis can usually be distinguished histologically from chronic idiopathic UC or Crohn colitis. Severe perianal disease, rectal sparing, absence of bleeding, and asymmetric or segmental involvement of the colon indicate Crohn disease rather than UC.

X-rays are not diagnostic but occasionally show abnormalities. Plain x-rays of the abdomen may show mucosal edema, loss of haustration, and absence of formed stool in the diseased bowel. Barium enema shows similar changes, albeit more clearly, and may also show ulcerations. A shortened, rigid colon with an atrophic or pseudopolypoid mucosa is often seen after several years of illness. X-ray findings of thumbprinting and segmental distribution are more suggestive of intestinal ischemia or possibly Crohn colitis rather than of UC.

As used herein, the term “graft” refers to organs and/or tissues which can be obtained from a first mammal (or donor) and transplanted into a second mammal (a recipient), preferably a human. The term “graft” encompasses, for example, skin, eye or portions of the eye (e.g., cornea, retina, lens), muscle, bone marrow or cellular components of the bone marrow (e.g., stem cells, progenitor cells), heart, lung, heart-lung (e.g., heart and a single lung, heart and both lungs), liver, kidney, pancreas (e.g., islet cells, β-cells), parathyroid, bowel (e.g., colon, small intestine, duodenum), neuronal tissue, bone and vasculature (e.g., artery, vein). A graft can be obtained from a suitable mammal (e.g., human, pig, baboon, chimpanzee), or under certain circumstances a graft can be produced in vitro by culturing cells, for example, embryonal cells, fetal cells, skin cells, blood cells and bone marrow cells which were obtained from a suitable mammal. A graft is preferably obtained from a human.

An “allograft”, as the term is used herein, refers to a graft comprising antigens which are allelic variants of the corresponding antigens found in the recipient. For example, a human graft comprising an MHC class II antigen encoded by the HLA-DRB1*0401 allele is an allograft if transplanted into a human recipient whose genome does not comprise the HLA-DRB1*0401 allele.

In one embodiment, the method of inhibiting (reducing or preventing) graft rejection comprises administering an effective amount of engineered T cells of the invention, in combination with targeting antibodies.

### Engineered Treg Cells

Embodiments of the invention include cells that express a CAR of the invention, which may be a CAR comprising an scFv that specifically binds to FRβ, particularly human FRβ; or may be a MabCAR that specifically binds to a non-endogenous antigenic moiety, e.g. FITC. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector or mRNA, has been introduced. Therefore, recombinant cells are distinguishable from naturally occurring cells which do not contain a recombinantly introduced nucleic acid. In embodiments of the invention, a host cell is a T cell, particularly a Treg cell. As discussed above, the engineered Treg cells can also comprise exogenous coding sequences for immunomodulatory proteins.

The cells can be autologous cells, syngeneic cells, allogeneic cells and even in some cases, xenogeneic cells. In many situations, one may wish to be able to kill the engineered Treg cells. For this purpose, one can provide for the expression of certain gene products in which one can kill the modified cells under controlled conditions, such as inducible suicide genes.

In particular cases an individual is provided with therapeutic Tregs engineered to comprise an FRβ specific CAR of the invention, where indirect binding CAR T cells are administered in combination with a targeting antibody. The cells may be delivered at the same time or at different times as another type of therapy. The cells may be delivered in the same or separate formulations as another type of therapy. The cells may be provided to the individual in separate delivery routes as another type of therapy. The cells may be delivered by injection at a lesion site, i.v., i.m., i.p., etc. Routine delivery routes for such compositions are known in the art.

Expression vectors that encode the mabCAR of the invention can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some embodiments AAV, retroviral or lentiviral vectors are used to deliver the CAR of the invention to a T cell. Adeno associated virus (AAV) is an attractive vector system for use in the cells of the present invention as it has a high frequency of integration and it can infect nondividing cells, thus making it useful for delivery of genes into mammalian cells, for example, in tissue culture or in vivo. AAV has a broad host range for infectivity. Details concerning the generation and use of rAAV vectors are described in U.S. Pat. Nos. 5,139,941 and 4,797,368, each incorporated herein by reference.

Retroviruses are useful as delivery vectors because of their ability to integrate their genes into the host genome, transferring a large amount of foreign genetic material, infecting a broad spectrum of species and cell types and of being packaged in special cell lines.

Lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2 and the Simian Immunodeficiency Virus: SIV. Recombinant lentiviral vectors are capable of infecting non-dividing cells and can be used for both in vivo and ex vivo gene transfer and expression of nucleic acid sequences. In some embodiments, the lentiviral vector is a third-generation vector (see, for example, Dull et al. (1998) J Virol. 72(11):8463-71). Such vectors are commercially available. 2nd generation lentiviral plasmids utilize the viral LTR promoter for gene expression, whereas 3rd-generation transfer vectors utilize a hybrid LTR promoter, see, for example Addgene for suitable vectors.

The cells may be administered as desired. Depending upon the response desired, the manner of administration, the life of the cells, the number of cells present, various protocols may be employed. The number of administrations will depend upon the factors described above at least in part.

### Methods

The Tregs that have been modified with the construct(s) are grown in culture under selective conditions and cells that are selected as having the construct may be expanded and further analyzed, using, for example; the polymerase chain reaction for determining the presence of the construct in the host cells. Once the modified host cells have been identified, they may then be used as planned, e.g. expanded in culture or introduced into a host organism.

Depending upon the nature of the cells, the cells may be introduced into a host organism, e.g. a mammal, including humans, in a wide variety of ways. The cells may be introduced at the site of the inflammatory lesion.

Targeting antibodies are administered to a subject prior to, or concurrent with, or after administration of mabCAR expressing T cells. The targeting antibodies bind to target cells in the subject, e.g. sites of inflammatory lesions. The targeting antibodies may be formulated for administered to a subject using techniques known to the skilled artisan. Formulations of the tagged proteins may include pharmaceutically acceptable excipient(s). Excipients included in the formulations will have different purposes depending, for example, on the nature of the tag, the protein, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

A formulation of targeting antibodies may include one type of targeting antibody, or more than one, such as two, three, four, five, six or more types of targeting antibodies. The different types of targeting antibodies can vary based on the identity of the antigenic moiety, the identity of the antibody, or both.

The targeting antibodies may be administered to a subject using modes and techniques known to the skilled artisan. Exemplary modes include, but are not limited to, intravenous, intraperitoneal, and intratumoral injection. Other modes include, without limitation, intradermal, subcutaneous (s.c., s.q., sub-Q, Hypo), intramuscular (i.m.), intra-arterial, intramedullary, intracardiac, intra-articular (joint), intrasynovial (joint fluid area), intracranial, intraspinal, and intrathecal (spinal fluids). Any known device useful for parenteral injection or infusion of the formulations can be used to effect such administration.

Formulations comprising the targeting antibodies are administered to a subject in an amount which is effective for treating and/or prophylaxis of the specific indication or disease. In general, formulations comprising at least about 0.1 mg/kg to about 100 mg/kg body weight of the tagged proteins are administered to a subject in need of treatment. In most cases, the dosage is from about 1 mg/kg to about 10 mg/kg body weight of the tagged proteins daily, taking into account the routes of administration, symptoms, etc.

In accordance with the present invention, a therapeutic composition of an engineered Treg cell expressing a mabCAR is administered as a therapy to a subject. The dose of Treg calls may be 10⁴, 10⁵, 10⁶, 10⁷, 10⁸, 10⁹ or more/kg body weight. The cells may be administered in any suitable excipient that maintains the viability of the cells.

In some embodiments, the subject has been diagnosed with T1D or IDDM, or pre-IDDM or pre-T1 D. One of skill in the art can determine the patients who would potentially benefit from a therapeutic agent that would reduce or prevent the development of overt diabetes. One of skill in the art can determine the therapeutically effective amount of the composition to be administered to a subject based upon several considerations, such as local effects, pharmacodynamics, absorption, metabolism, method of delivery, age, weight, disease severity and response to the therapy.

An improvement in diabetic parameters may be any observable or measurable improvement. Thus, one of skill in the art realizes that a treatment may improve the patient or subject's condition, but may not be a complete cure of the disease. In certain aspects, the composition is administered in an effective amount to decrease, reduce, inhibit or abrogate levels of immune response from the donor's cells, tissue and/or organ against the host's tissues.

Efficacy may be monitored in a patient during treatment, e.g. by monitoring weight for a reduction in obesity, where weight loss of at least about 5%, at least about 10%, at least about 15% or more can indicate successful treatment. Measurement of insulin sensitivity is another relevant marker for monitoring efficacy, where an increase in M value may be at least about 10%, at least about 25%, at least about 50% or more with successful therapy, for example normal values for a non-insulin resistant subject may be at least about 8, at least about 9, at least 10 or higher.

In an embodiment of the present invention, the engineered Treg composition is administered in an effective amount to decrease, reduce, inhibit or abrogate inflammation of the pancreas and toxicity related to standard therapy, in combination with an effective dose of targeting antibodies. The amount of antibody in the composition may vary from about 1 ng to about 1 g, more preferably, 0.1 mg to about 100 mg.

Treatment regimens may vary as well, and often depend on the health and age of the patient. Certain types of disease will require more aggressive treatment, while at the same time, certain patients cannot tolerate more taxing regimens. The clinician will be best suited to make such decisions based on the known efficacy and toxicity (if any) of the therapeutic formulations.

In specific embodiments, the composition is given in a single dose or multiple doses. The single dose may be administered daily, or multiple times a day, or multiple times a week, or monthly or multiple times a month. A series of doses may be administered daily, or multiple times a day, weekly, or multiple times a week, or monthly, or multiple times a month.

The improvement is any observable or measurable improvement. Thus, one of skill in the art realizes that a treatment may improve the patient or subject's condition, but may not be a complete cure of the disease. In certain aspects, the composition is administered in an effective amount to decrease, reduce, inhibit or abrogate levels of an immune response against the recipient.

In order to increase the effectiveness of oral administration of the composition of the present invention, these compositions may be combined with conventional therapy.

The composition of the present invention may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

Various combination regimens of the composition and one or more agents are employed. One of skill in the art is aware that the composition of the present invention and agents can be administered in any order or combination.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment. To prepare pharmaceutical compositions, an effective amount of the antibody may be dissolved or dispersed in a pharmaceutically acceptable carrier or aqueous medium. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antibody can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils.

The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with various of the other ingredients enumerated above, as required, followed by filtered sterilization.

Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose.

These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The targeting antibodies may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means. Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

All references cited in this specification are hereby incorporated by reference in their entirety. The following examples are solely for the purpose of illustrating one embodiment of the invention.

The invention will further be illustrated in view of the following figures and example.

### EXPERIMENTAL

Current immunosuppressive medications such as steroids, cyclosporine or blocking monoclonal antibodies have broad activity and are not localized to sites of inflammation. These agents all act by blocking immune cell activation signals. A fundamentally different approach is to use regulatory T cell therapy (Tregs) cell therapy to treat immune diseases. Tregs actively suppress immune responses and do not just block activation of immune cells. Tregs in pre-clinical models and a few clinical trials have effectively controlled dysregulated immune responses. However, major challenges to the clinical implementation of Tregs remain: (a) Tregs must be more effectively targeted to tissue sites of immune attack, (b) Tregs must be better insulated against inactivation by inflammatory cytokines, and (c) Tregs must be better controlled from being too active for too long.

Recent advances in the genetic modification of T cells have led to promising cellular therapies, including those with engineered chimeric antigen receptor (CAR) T cells. In CAR T cell therapy, DNA constructs are transduced into T cells usually using viral vectors. A CAR fusion protein is expressed that possesses a surface antibody-binding domain and an internal cell-signaling domain. T cells are thus “re-wired” to recognize and destroy defined surface antigens such as the B cell surface receptor CD19. CAR T cells have proven very effective in treating otherwise untreatable B cell leukemia and lymphoma.

There are different types of Tregs, including the well described CD25⁺ FOXP3⁺ natural subset (nTreg). nTregs are enhanced by transducing them to express engineered CAR that have a surface domain that recognizes fluorescein conjugated to the Fc “back-end” of mAbs.

### Example 1

### The Use of Chimeric Antigen Receptor T Regulatory Cells Reduces Insulin Resistance and Colitis in Mice

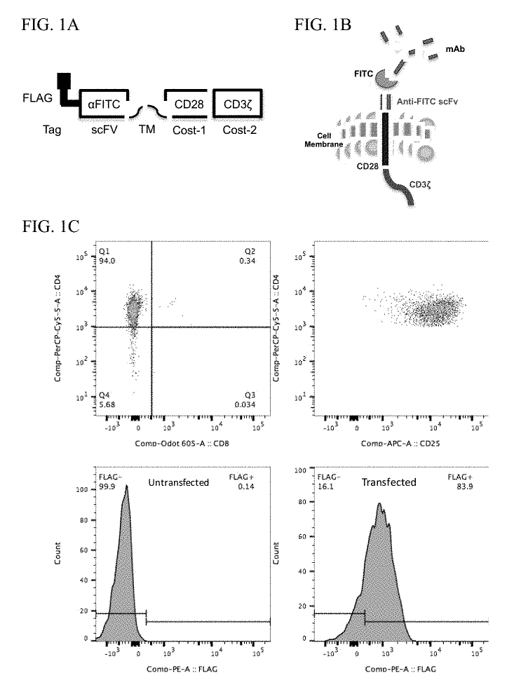

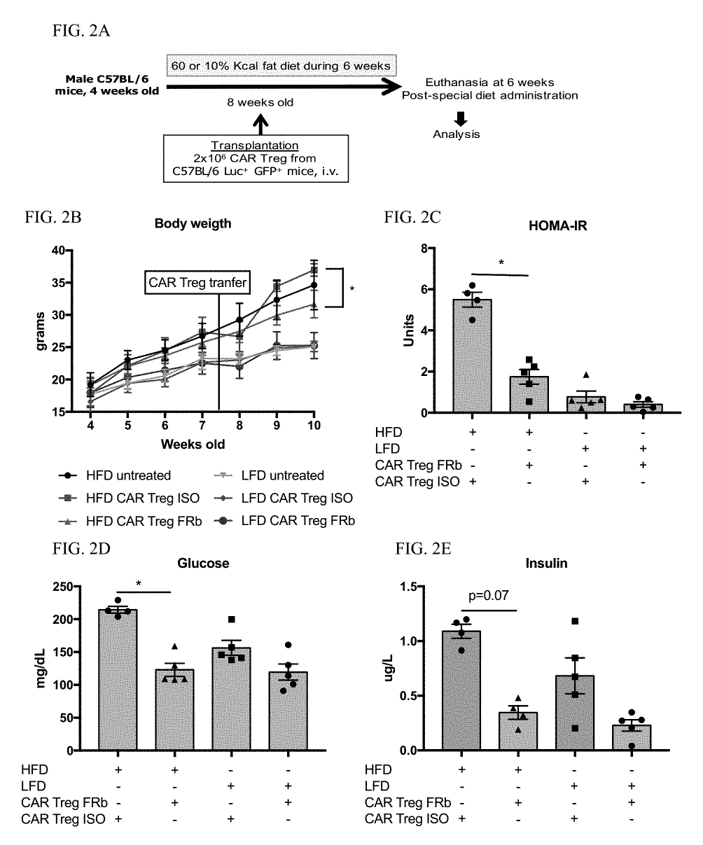

Chimeric antigen receptor T regulatory therapy reduce obesity and insulin resistance in mice. The treatment of chimeric antigen receptor (CAR) Treg targeted with monoclonal FITC anti-folate receptor b antibody, which is expressed in different tissues, for example epithelial cells and macrophages, was used to assess the impact of T cell therapy in type 2 diabetes. High- or low-fat diet (HFD or LFD) were provide to male C57BL/6 mice 4 weeks old during 6 weeks. After 4 weeks of special diet, mice were treated or not with CAR Treg targeted with FITC anti-Folate receptor b or isotype control antibody (2×10⁶ cells/mouse; i.v.). Mice received a dose of CAR Treg (FIG. 1 and FIG. 2A).

HFD mice increased their body weight significantly during the 6 weeks. Any difference was shown between HFD controls or HFD isotype control antibody treated mice. In contrast, body weight was significantly decreased in obese mice after CAR Treg anti-folate receptor b therapy. Low fat diet significantly reduced body weight in mice respect to HFD mice (FIG. 2B).

Moreover, obese mice shown that CAR Treg anti-folate receptor b therapy protect the recipient mice against insulin resistance (FIG. 2C). However, CAR treg control isotype antibody therapy failed to protect the recipient mice against insulin resistance and further reduced the body weight (FIG. 2B-C).

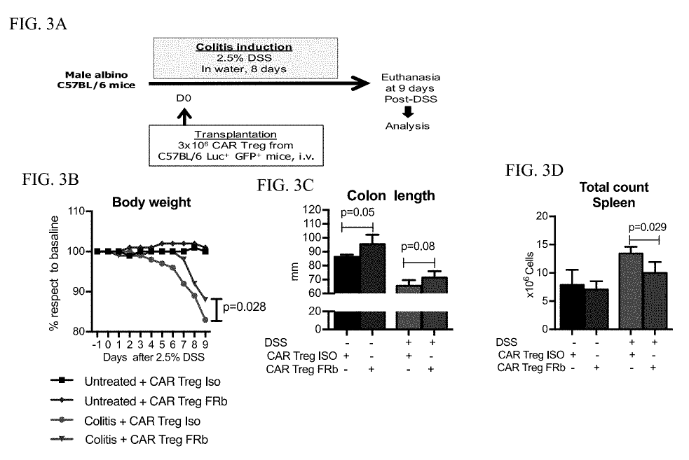

Chimeric antigen receptor T regulatory therapy prevent colitis and reduce inflammation in mice. The treatment of CAR Treg targeted with monoclonal FITC anti-folate receptor b antibody, was used also to assess the impact of T cells therapy in colitis. Dextran sulfate sodium salt (DSS) were provide to male C57BL/6 mice 10 weeks old during 8 days. Mice were treated or not with CAR Treg targeted with FITC anti-Folate receptor b or isotype control antibody (3×10⁶ cells/mouse; i.v.). Mice received a dose of CAR Treg (FIG. 1 and FIG. 3A).

DSS-treated mice decrease their body weight significantly after DSS treatment. Any difference was shown between CAR Treg ISO controls or CAR Treg Folate receptor b in untreated mice. In contrast, body weight was significantly decreased in DSS-treated mice after CAR Treg anti-ISO therapy. CAR Treg anti-folate receptor b significantly reduced body weight loss in DSS-treated mice respect to DSS CAR Treg ISO-treated mice (FIG. 3B).

Moreover, DSS-treated mice shown that CAR Treg anti-folate receptor b therapy protect the recipient mice against colitis and inflammation in spleen (FIG. 3C-D). However, CAR Treg control isotype antibody therapy failed to protect the recipient mice against colitis (FIG. 3C-D).

### Materials and Methods

Cell isolation. Regulatory T cells were isolated from spleens and lymph nodes cell suspension, stained for CD25-allophycocyanin (APC) and CD4, enriched with anti-APC MACS beads and sorted for CD4⁺CD25⁺GFP⁺ cells from C57BL/6 albino FoxP3luc⁺GFP⁺ mice on a FACS Verdi (BD Biosciences, San Jose, Calif.). Purity of the final Treg product was always >95% CD4⁺FoxP3⁺ cells.

In vitro cell culture. Treg were plated in 96-well or 48-well flat-bottom plates containing RPMI, interleukin-2 (IL-2, 1000 IU/ml), 10% Fetal bovine serum (FBS), 1% L-Glutamine, 1% Penicillin, Streptomycin and anti-CD3/CD28 beads (Dynabeads, Invitrogen, 1:1 bead:cell ratio for Treg). Treg have been cultured for 20 days, checked for purity by FACS analysis (CD4⁺CD25⁺FoxP3⁺ cells constantly >90%), washed, and then transfected.

MabCAR construct. All lentiviral constructs are based on the self-inactivating pHR vector.

The sequence of the scFv 1x9Q is as follows:

(SEQ ID NO: 2)
ASDVVMTQTPLSLPVSLGDQASISCRSSQSLVHSNGNTYLRWYLQKPG
QSPKVLIYKVSNRVSGVPDRFSGSGSGTDFTLKINRVEAEDLGVYFCS
QSTHVPWTFGGGTKLEIKSSADDAKKDAAKKDDAKKDDAKKDGGVKLD
ETGGGLVQPGGAMKLSCVTSGFTFGHYWMNWVRQSPEKGLEWVAQFRN
KPYNYETYYSDSVKGRFTISRDDSKSSVYLQMNNLRVEDTGIYYCTGA
SYGMEYLGQGTSVTVSS.

Following is the transmembrane domain and signaling domains of murine CD28 and CD3 zeta:

(SEQ ID NO: 3)
“IEFMYPPPYLDNERSNGTIIHIKEKHLCHTQSSPKLFWALVVVAGVL
FCYGLLVTVALCVIWTNSRRNRGGQSDYMNMTPRRPGLTRKPYQPYAP
ARDFAAYRPRAKFSRSAETAANLQDPNQLFNELNLGRREEFDVLEKKR
ARDPEMGGKQQRRRNPQEGVYNALQKDKMAEAYSEIGTKGERRRGKGH
DGLFQGLSTATKDTFDALHMQTLAPR”

A MabCAR for human use is similar to the murine 1 X9Q construct. The sequence of 1X9Q is the same and the signaling domains are replaced by human CD28 and CD3 zeta.

Treg Transfection and Incubation with FITC-Conjugated Antibody.

Treg were transfected by electroporation using Lonsa system using mRNA. Briefly, cells were washed with PBS 2% FBS and removed Beads CD3/CD28 beads with magnetic support plated. 10×10⁶ cells by cubete were incubated with the 1X9Q RNA as well as the mouse T cell nucleofector solution. Transfected cells were then cultured over night to allow for expression of the chimeric receptor. Cells were incubated or not in vitro with the FITC conjugated antibody of interest for 30 minutes on ice.

FITC conjugated antibodies that have been used for stimulation of mAbCAR. mAbCAR Treg are the followings: Anti-Folate Receptor β (CL10, Absolute antibody), IgG1 kappa (MOPC-21, Biolegend). Transfection efficiency was checked by Flow cytometry using Anti-Flag antibody PE and Zombie Aqua™ Dye (Biolegend). Cells were then washed once more and injected into mice. CAR Treg targeted with Anti-Folate receptor b or isotype control antibody were given at indicated time point (FIG. 1A). Control animals were either untreated. The anti-folate receptor antibody is described in Nagai et al. (2009) Cancer Immunol Immunother. 58(10):1577-86.

Mice. Male four-weeks old C5BL/6 mice were purchased from Jackson Laboratories (Sacramento, Calif.). Mice were maintained under a 12-hours light-dark cycle and they were fed with a standard laboratory diet. All studies were approved by Institutional Animal Care and Use Committee of Stanford University.

Induction of insulin resistance in obese mice as a type 2 diabetes model. Obesity was induced by the ad libitum administration of especial high-fat diet (HFD) or low-fat diet (LFD) as control (60% or 10% kcal respectively; Open Source Diets) for 6 weeks. Animals were monitored daily for behavior, aspect alteration for a period of 5 weeks. Body weight, glucose blood levels and insulin resistance were measure weekly. Animals presenting signs of total immobility were instantaneously euthanized by CO₂ atmosphere and cervical dislocation. CAR-Treg targeted with anti-folate receptor B or isotype control antibody were given at indicated time points (FIG. 2A).

Induction of Acute DSS-Induced Colitis. Colitis was induced by the administration of DSS (molecular weight, 40 kilodaltons; Sigma Aldrich). 2.5% DSS were dissolved in sterile, distilled water ad libitum for 8 days. Fresh DSS solution was prepared daily followed by normal drinking water. Mice were sacrificed at day 9 following colitis induction. Control animals were either untreated. Animals presenting signs of suffering (weight loss >20%, prostration, tremors) were instantaneously euthanized by cervical dislocation. The entire colon was removed from the caecum to the anus, then measured. CAR-Treg targeted with anti-folate receptor B or isotype control antibody were given at indicated time points (FIG. 3A).

Sample size and statistical analysis. The statistical analysis was performed using Prism 5 (GraghPad software, San Diego, Calif.). Results were analyzed using non-parametric test (Mann Whitney tests), T test expressed in terms of probability (P). Differences were considered significant when p<0.05. All data were expressed as mean±SEM.

### Example 2

### Targeted CAR-Treg Promote Increased Chimerism after Hematopoietic Stem Cell Transplantation

The role of chimeric antigen receptor (CAR) regulatory T cells (CAR-Treg) in promoting tolerance after hematopoietic stem cells transplantation leading to hematopoietic mixed chimerism was investigated. Hematopoietic cell chimerism may facilitate donor-specific tolerance to transplanted organs as well as eliminating the need for immunosuppressive therapy.

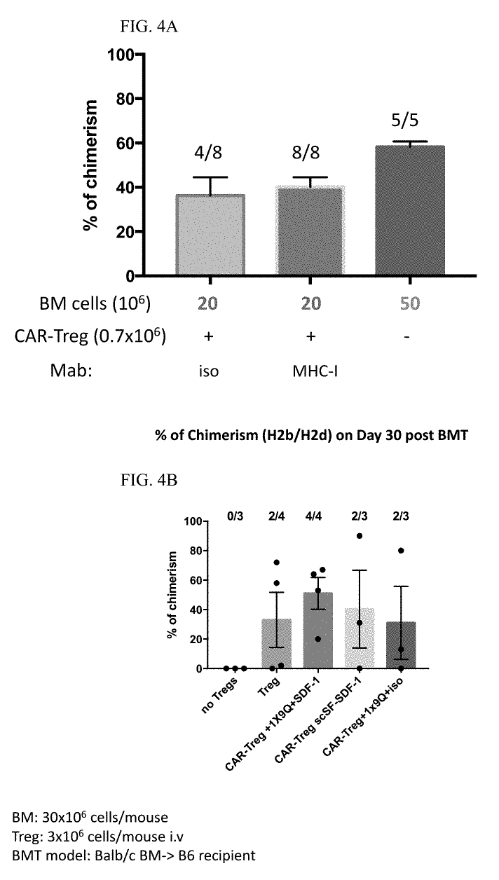

To address this question experimentally, 057/B6 (B6) mice received 10 doses of total lymphoid irradiation (TLI) in addition to 5 doses of ATS, a well-established regime that conditions mice to receive allogeneic bone marrow (BM) cells from Balb/c mice. In addition to BM cells, mice also received CAR-Tregs that express a chimeric antigen receptor, which recognizes FITC, as described in Example 1. Before inoculation into mice, CAR-Tregs were coated with either FITC-MHC-I mAb or a FITC-isotype control mAb. As seen in FIG. 4, mice that received CAR-Tregs coated with FITC-MHC-I mAb in addition to 20×10^{\hat{ }6} BM cells showed 2-fold increased incidence of chimerism compared to the mice that received CAR-Tregs coated with the isotype control mAb, even thought there was no difference at the levels of chimerism observed within the two groups.

In addition, under another conditioning regime where mice received sublethal irradiation of 700 cGy instead of TLI/ATS, we observed increased chimerism in the group that received CAR-Treg coated with FITC-MHC-I mAb as opposed to the control group that received CAR-Treg coated with FITC-isotype control mAb (Table 1).

TABLE 1

| | | | Number of | |
|---|---|---|---|---|
| Group | Tregs | FITC-Ab | chimeric animals | % of chimerism |
| A | − | — | 0/5 | 0 |
| B | + | iso | 0/5 | 0 |
| C | + | MHC-I | 1/5 | 32% |

Besides MHC-class I mAb as a target to CAR-Tregs, we explore the efficacy of another molecule, the chemokine stromal derived factor-1 (SDF-1). SDF-1 has been implicated in hematopoietic stem cells migration and homing in the BM, as well as in trafficking of Treg cells to the hematopoietic niche. As shown in FIG. 4, mice that received CAR-Treg coated with FITC-MHC-1 and FITC-SDF-1 mAbs in addition to allogeneic BM cells showed higher levels of chimerism, as opposed to the control group that received CAR-Treg coated with FITC-isotype control mAb.

Other targets for CAR-Tregs homing include integrin VLA-4, VLA-5 and Rac-1, molecules that have demonstrated essential role in BM homing of stem/progenitor cells, as well as the folate receptor.

What is claimed is:

1. A method for treating an inflammatory condition in a subject in need thereof, comprising administering to said subject:
an effective dose of regulatory T cells (Treg) engineered to express a chimeric antigen receptor (CAR), that specifically binds folate receptor beta (FRβ);
wherein inflammation is decreased at the targeted site.

2. The method of claim 1, wherein the CAR directly binds to FRβ.

3. The method of claim 1, wherein the CAR specifically binds to a small molecule non-endogenous antigenic moiety; and is administered in combination with an effective dose of targeting antibodies, which antibodies (i) bind to FRβ and (ii) are labeled with the non-endogenous antigenic moiety.

4. The method of claim 3, wherein the antigenic moiety is fluorescein isothiocyanate (FITC).

5. The method of claim 1, wherein the Treg cells are isolated from a peripheral blood sample.

6. The method of claim 1, wherein the Treg cells are expanded in culture.

7. The method of claim 1, wherein the subject is a human.

8. The method of claim 1, where the inflammatory condition is diabetes.

9. The method of claim 8, wherein the diabetes is Type 2 diabetes.

10. The method of claim 9, wherein obesity is reduced following administration of the regulatory T cells.

11. The method of claim 9, wherein insulin resistance is decreased following administration of the regulatory T cells.

12. The method of claim 1, wherein the inflammatory condition is inflammatory bowel disease.

13. The method of claim 12, wherein colitis is decreased following administration of the regulatory T cells.

14. A cellular composition of engineered Treg cells for use in the method of claim 1.

15. A kit comprising the cellular composition of claim 14, and a suitable targeting antibody.

16. A method for enhancing chimerism of a recipient following hematopoietic cell transplantation (HCT), comprising:
administering to said subject an effective dose of regulatory T cells (Treg) engineered to express a chimeric antigen receptor (CAR), that specifically binds a protein involved in hematopoietic stem cell migration and/or homing; in combination with an effective dose of hematopoietic stem cells.

17. The method of claim 16, wherein the transplant recipient has been treated with myeloablative conditioning regimen prior to administering the Treg and hematopoietic stem cells.

18. The method of claim 16, wherein the protein involved in hematopoietic stem cell migration and/or homing is SDF-1.

19. The method of claim 16, wherein the protein involved in hematopoietic stem cell migration and/or homing is MHC Class I protein.

20. The method of claim 16, wherein the hematopoietic cell sample is obtained from bone marrow, from cord blood, or by apheresis from mobilized peripheral blood.


SEQUENCE LISTING (3 sequences)

SEQ ID NO: 1 - protein, 5 aa, Artificial Sequence
  Synthetic sequence
  Misc_feature (1)..(5): This stretch of amino acid residues may be repeated
EAAAK

SEQ ID NO: 2 - protein, 257 aa, Artificial Sequence
  Synthetic sequence
ASDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSS

SEQ ID NO: 3 - protein, 217 aa, Artificial Sequence
  Synthetic sequence
IEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR